(19) United States
(12) Patent Application Publication
(10) Pub. No.: US 2015/0193560 A1
(43) Pub. Date: Jul. 9, 2015

(54) METHOD FOR CREATING CUSTOM ORTHOPEDIC SUPPORTS FROM COMPUTERIZED DATA INPUTS

(71) Applicant: Donald SPECTOR, New York, NY (US)

(72) Inventor: Donald SPECTOR, New York, NY (US)

(21) Appl. No.: 14/666,412

(22) Filed: Mar. 24, 2015

Related U.S. Application Data
(63) Continuation-in-part of application No. 14/030,081, filed on Sep. 18, 2013, now Pat. No. 9,020,626.
Application No. 14/030,081 is a continuation of application No. 11/737,454, filed on Apr. 19, 2007, now Pat. No. 8,583,272.
Application No. 11/737,454 is a continuation-in-part of application No. 11/408,769, filed on Apr. 21, 2006, now abandoned.

(51) Int. Cl. G06F 17/50; A61F 5/01; B29C 67/00

(52) U.S. Cl. CPC G06F 17/50; B29C 67/0088; A61F 5/01

(57) ABSTRACT

Systems and methods of measuring feet and designing and creating orthopedic inserts are described. A leg length discrepancy of a user is measured and this data, along with a shoe model and size are input into a computer. The computer has stored information regarding foot bed dimensions of various shoe models and sizes and matches the input shoe model with the stored information. The information regarding the selected shoe model is used along with the leg length discrepancy to create a computer model of a custom shoe insert to be used with the selected shoe. The computer model is then sent to a 3D printer to print the insert.

### STATEMENT OF RELATED CASES

This application is a continuation in part of U.S. patent application Ser. No. 14/030,081, which is a continuation of U.S. patent application Ser. No. 11/737,454, filed on Apr. 19, 2007, which is a continuation-in-part of U.S. patent application Ser. No. 11/408,769, filed Apr. 21, 2006, all of which are incorporated herein by reference.

### BACKGROUND OF THE INVENTION

This product relates to the field of orthopedic inserts for shoes, sneakers and other footwear.

These product lines are typically divided into two categories. One such category is defined by relatively universal insoles that cushion and provide general support. A previous patent discloses the first adjustable insole, in which the bounce of the insole can be controlled by turning a valve. Other products on the market are those such as manufactured under the brand name Dr. Scholl's.

Most of these products tend to be for comfort or support and are universal in use. They are relatively inexpensive.

At the other end of the spectrum are the devices referred to as supports. These are often made by Podiatrists. Podiatrists take imprints and casts of people's feet and then have inserts designed that are made to correct the weight and imprint of the foot.

Accordingly, new and improved methods and systems to provide orthopedic insets are needed.

### SUMMARY OF THE INVENTION

One aspect of the present invention is a method of measuring a foot and developing an orthopedic insert to correct for leg length discrepancy (LLD). This insert can then be attached to shoes or other inserts which correct for other foot problems, such as over- or under-pronation, plantar fasciitis, etc. The invention includes measuring the leg length discrepancy using any desired method, and manufacturing the orthopedic insert based on the design. In particular, the insert can be produced to exactly match a particular shoe, so that each insert is custom made for a particular shoe design.

The system, in accordance with one aspect of the present invention, includes measuring leg length discrepancy and communicating the data regarding the leg length discrepancy to a computer that then designs an orthopedic insert for printing on a 3D printer. The analysis and the making of the orthopedic insert are performed automatically after measuring the length. In addition using leg length discrepancy data, data regarding a particular shoe model is also used to design a custom insert that fits perfectly within a particular shoe. The computer stores footbed data for a series of different shoe models so that the insert is printed with the exact footprint of the desired shoe.

The leg length discrepancy can be measured using any suitable measurement method, such as a tape measure, photography, X-ray or laser scanning.

### DESCRIPTION OF THE DRAWINGS

FIG. 1 illustrates the system according to the invention;

FIG. 2 illustrates a shoe insert created with the method according to the invention;

FIG. 3 illustrates a shoe having a custom insert according to the invention; and

FIG. 4 illustrates the method steps for creating a shoe insert according to the invention.

### DESCRIPTION OF THE EMBODIMENTS

One object of this invention to create a collateral informational base that is immediately capable of altering the manufacture of inserts on a personal basis in an economic way. This can be particularly important since the cost of inserts by podiatrists typically cost hundreds of dollars, for an end product that probably has a cost of goods of only a few dollars. The computerized instructions for the manufacturing of the insert can be transmitted to a foreign country where these inserts can be made by hand or eventually transmitted to machines that can actually create three dimensional moldings that can then be sold to the consumers at a faction of the cost of present inserts sold by Podiatrists. The insert according to the invention can also be attached to existing custom or mass produced orthotics to compensate for leg length discrepancies. The insert could also be attached directly to a shoe.

It is believed that almost 90% of the population has two different size legs. Typically, people with two different size legs learn to compensate for this difference by favoring one side. Experts in the field have shown that this is a primary cause of scoliosis, back problems, hip problems as well as pain in later life. This problem can be immediately eliminated using instant 3D foot inserts in accordance with the various aspects of the present invention.

Traditionally, people have gone to podiatrists to make castings, and from these, to make inserts. This process costs several hundred dollars and requires multiple visits. Most people do not go through this effort and expense, unless they have severe problems. However, the chronic problems do not manifest as major problems until later, and are not detected until permanent damages has been done, for example, to the spinal column.

The present invention offers a simple easy technology based on 3D printers that are connected to a computer platform to instantly read and identify problems with weight distribution and the size of a person's leg to automatically print out a plastic insert without the need for casts, multiple visits or large expense.

Referring to FIG. 1, one embodiment of the present invention is illustrated. A person 100 has two legs 102 and two feet 104. The length of the legs 102 is measured and determined using a measuring device 106. The measuring device can be a laser or any other type of measuring device. For example, a simple tape measure could even be used. The length of the legs 102 or the discrepancy between the length of the legs is entered into a processor 108. Also entered into a processor 108 is a specific model of shoe for which the insert is to be used, as well as the shoe size. Processor 108 is connected to a database 110 that stores foot bed data for various shoe models and sizes. Processor 108 stores software that is configured to generate a computer model of a custom orthopedic insert that precisely fits the selected shoe model and size, as well as compensates for any leg length discrepancy.

The information concerning the orthopedic inserts are transmitted from the processor 108 to a 3D printer 112, also known as a rapid prototyping machine. The 3D printer then instantly generates the necessary orthopedic insert or inserts 114. Insert 114 is manufactured to fit a particular shoe model exactly, and is custom made to compensate for the user's leg length discrepancy as well. Thus, the shoe insert 114 is custom made for both the user and the shoe. The method of the present invention can be performed by a technician entering the leg length discrepancy information and shoe model information directly into a computer connected to the 3D printer, or can take place remotely. In the remote situation, a customer could log on to a remote server via the internet, and enter in the shoe model information and leg length discrepancy measurements into a template on the server. This information can then be used for configuration of the computer model and sent to the printer for printing the insert. The printer can be directly connected to the server or can be located remotely. In the remote situation, the printer is connected to a processor which processes the information received from the server to configure the insert for printing. The server sends the data over the internet to the processor which then creates the computer model of the insert using the received data.

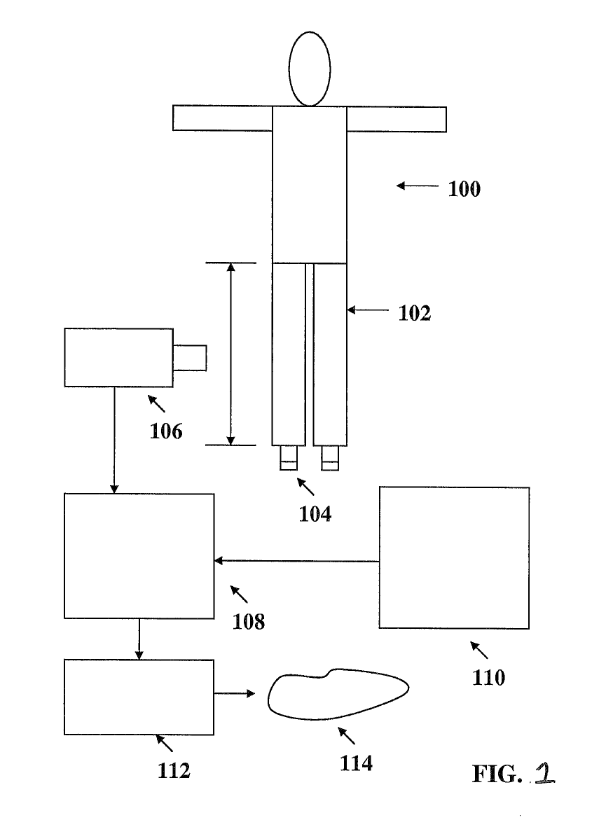

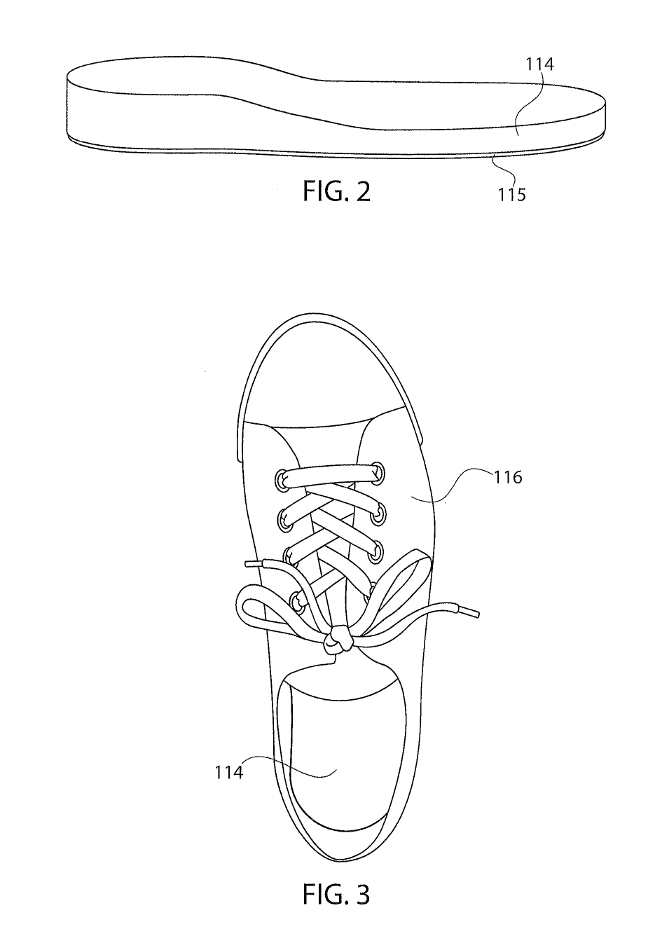

FIG. 2 shows the insert 114 according to the invention. Insert 114 is supplied with an adhesive layer 115 for attachment to a particular shoe 116, shown in FIG. 3. In this case, shoe 116 is model number 11671, which is transmitted to the processor 108 so a corresponding insert 114 is printed that exactly matches the shoe model and size.

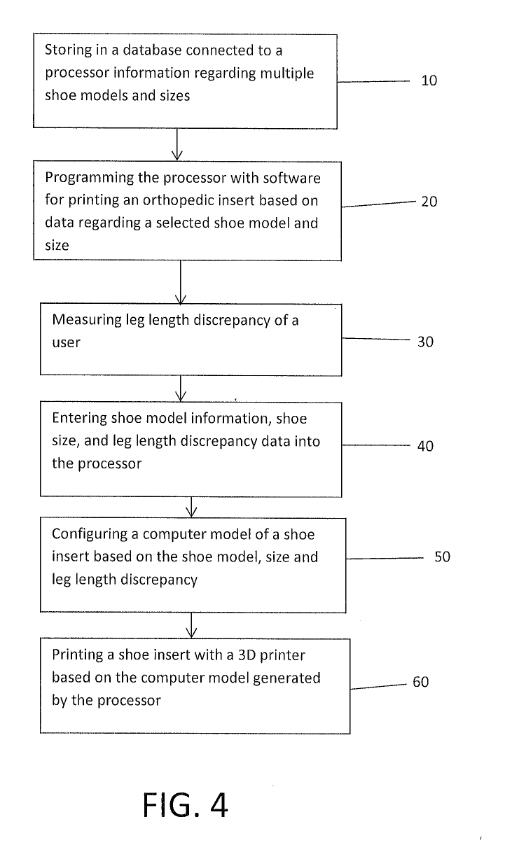

FIG. 4 illustrates the method steps according to the invention. In a first step 10, information regarding various shoe models and sizes is stored in a database connected to a processor. The information stored is relates to the size and shape of the particular footbed of each shoe model. Next, in step 20, the processor is programed with software for printing an orthopedic insert based on data input by a user regarding the thickness of the insert and the particular shoe model and size.

Next, in step 30, a user measures a leg length discrepancy using any desired method: tape measure, X-ray, laser, etc. The amount of leg length discrepancy is entered into processor 108 by the user in step 40, along with the desired shoe model indentifier and size.

In step 50, processor 108 takes the leg length discrepancy data and shoe model and size data and configures a computer model for a custom insert for the user.

In step 60, this computer model is then sent to printer 112 for printing the actual insert. Thus, a completely custom insert can be made quickly and inexpensively. Each shoe can have its own insert that fits precisely within the shoe.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

What is claimed is:

1. A method of developing at least one orthopedic insert for footwear used by a person, the method comprising:
storing in a computer data regarding foot bed measurements of a number of shoe models and sizes;
receiving in the computer a data input defining a leg length discrepancy of the person, such input determined by a measuring device;
receiving in the computer a data input regarding a particular shoe model and size;
matching the received data input regarding the particular shoe model and size with one of the stored foot bed measurements;
designing in the computer via a processor a computer model of an orthopedic insert based on the input leg length discrepancy data and the foot bed measurement matching the data input regarding the particular shoe model and size;
transmitting the computer model of the orthopedic insert from the computer to manufacturing device; and
making the orthopedic insert with the manufacturing device based on the computer model.

2. The method according to claim 1, further comprising the steps of applying an adhesive to one surface of the insert.

3. The method according to claim 1, wherein the steps of receiving include receiving the data regarding leg length discrepancy and shoe model and size via a transmission over the internet.

4. The method according to claim 1, wherein the manufacturing device is a 3D printer.
